(19) United States
(12) Patent Application Publication
(10) Pub. No.: US 2012/0059308 A1
(43) Pub. Date: Mar. 8, 2012

(54) PERIPHERAL NERVE BLOCK CATHETER

(75) Inventors: Hawpeng S. Hsu, St. Louis, MO (US); Chris C. Lee, St. Louis, MO (US)

(73) Assignee: THE WASHINGTON UNIVERSITY, St. Louis, MO (US)

(21) Appl. No.: 13/226,860

(22) Filed: Sep. 7, 2011

Related U.S. Application Data
(60) Provisional application No. 61/380,548, filed on Sep. 7, 2010.

(51) Int. Cl. A61M 19/00; A61N 1/36; B23P 17/04; A61M 25/00

(52) U.S. Cl. 604/21; 604/528; 607/2; 29/428

(57) ABSTRACT

An anesthetic nerve block catheter and methods of using the anesthetic nerve block catheter to perform a nerve block or continuous nerve block procedure are disclosed.

### CROSS-REFERENCE TO RELATED APPLICATIONS

This application claims benefit to U.S. Provisional Patent Application Ser. No. 61/380,548 filed on Sep. 7, 2010, the contents all of which is incorporated by reference.

### FIELD

The present disclosure generally relates to regional anesthetic nerve block catheters and methods of using the catheters to conduct regional anesthesia and analgesia, and in particular to an echogenic, kink resistant anesthetic nerve block catheters and a one-step method of situating such catheters for use in an anesthetic nerve block procedure.

### BACKGROUND

Regional anesthesia, including anesthetic nerve blocks are administered to ever-increasing numbers of patients during surgery, for the relief of post-operative pain, and for the extended relief of chronic pain as a safer and more affordable alternative to general anesthesia. The use of nerve blocks may be indicated over the use of general anesthesia because of increased safety and patient satisfaction, excellent post-operative pain control, and decreased anesthesia costs. Nerve blocks generally provide regional anesthesia by the introduction of a local anesthetic compound in close proximity to a sensory and/or motor nerve, thereby causing the inhibition of sensory or motor impulses travelling along the treated nerve. For example, a local anesthetic may be introduced near the femoral nerve in preparation for the performance of a total knee replacement surgery. The local anesthetic may be introduced for the duration of the surgery, or the anesthetic may be supplied for an extended period in order to manage pain during a recovery period, or even indefinitely in the case of a chronic pain disorder.

Existing nerve block techniques typically involve the location of the nerve that provides sensory and/or motor functions to an afflicted area such as an arm or leg. Once the nerve has been located, local anesthesia is introduced. If the nerve block is of short duration, a single injection from a needle may be sufficient. However, if the nerve block is to be of a more extended or indefinite duration, a catheter may be placed in order to continuously supply local anesthetic to the vicinity of the nerve to be blocked. At the conclusion of surgery, or if no further nerve blocking is required for pain management, the needle or catheter may be removed.

The nerve to be blocked may be located either by direct visualization techniques such as X-ray or ultrasound imaging, or through indirect techniques using a needle to probe for the nerve. In one indirect technique, the needle may be manipulated until parathesia, as defined herein to be a buzzing or tingling sensation that is reported by the patient to the practitioner. In a second indirect technique, an electrostimulation device is electrically connected to the probing needle, which is manipulated until a sensory or motor response is elicited from the patient.

Although existing techniques of locating a nerve to be blocked described above have been used by practitioners with limited success, each technique also imposes limitations on their effective use. For example, X-ray imaging may be impractical due to the relative scarcity of X-ray imaging equipment in an operating room setting. In addition, the positioning of the needle using X-ray imaging as a guide may be difficult to accomplish and may expose the patient and surgical team to unhealthy amounts of X-ray radiation. Ultrasound imaging is much more commonly available and considerably safer than X-ray imaging, but the relatively poor contrast of the needle relative to the surrounding tissues in the ultrasound image poses a challenge to all but the most experienced practitioners. Probing until parathesia is reported relies upon the accurate assessment and reporting of parathesia on the part of the patient, who may be under the influence of sedatives or may be otherwise unable to furnish reliable information. Although the use of electrostimulation does not require any active participation on the part of the patient, the patient's motor responses may affect the positioning of the needle, which may pose a challenge with respect to the accurate introduction of a catheter.

The anesthesia nerve block catheter, if used, is typically introduced through the lumen of the needle used to locate the nerve, and then the needle is removed. Because the needle is of a larger diameter than the catheter, local anesthesia may leak out from the space left between the outer wall of the catheter and inner surface of the entry wound created by the needle. This increases the amount of anesthesia required to induce a desired effect, and introduces uncertainty as to how much anesthesia is actually introduced into the desired location near the blocked nerve. Additional uncertainty is introduced by existing techniques because the location at which the catheter is actually installed may differ significantly from the preferred location near the nerve due to movement of the locating needle during the process, which typically involved several separate steps. In addition, the existing catheter that is disposed inside the needle typically has a very small lumen, thereby making it highly resistant to injection or infusion of a local anesthetic into the lumen. If the catheter fails during removal, existing catheters are fabricated from materials that are not typically visible through common imaging techniques such as X-ray or ultrasound, making it very difficult to reposition the catheter or recover any catheter fragments inadvertently left in the patient's tissues.

A need exists for an improved nerve block catheter and method of positioning, inserting and repositioning the nerve block catheter. Such a catheter should exploit the advantages of existing nerve block catheters and methods, while minimizing the disadvantages described above. Such a nerve block catheter would enhance the safety of nerve block anesthesia by improving the accuracy of catheter placement, the reliability of the catheter once installed for brief or extended time periods, and simplify the training of practitioners unfamiliar with this anesthetic technique.

### SUMMARY

In one embodiment, a catheter may include an elongated sheath defining a central lumen in communication with a proximal end and a distal end, an echogenic material made from a biocompatible material surrounding the central lumen, and a connection fitting attached to the proximal end of the elongated sheath.

In another embodiment, a method of using a catheter may include:

providing a catheter may include:
an elongated sheath defining a central lumen in communication with a proximal end and a distal end,
an echogenic material made from a biocompatible material surrounding the central lumen, and
a connection fitting attached to the proximal end of the elongated sheath;
inserting an electro-stimulation needle comprising a beveled end and a conductive wire electrically connected at an opposite end thereof through the central lumen such that the beveled end protrudes outwardly from the distal end of the central lumen; and
puncturing a body wall with the beveled end of the electro-stimulation needle;
positioning the catheter such that the beveled end is adjacent or proximate a nerve; and
introduce stimulating electrical currents through the electro-stimulation needle.

In yet another embodiment, a method for manufacturing a catheter may include:
forming an elongated sheath defining a central lumen with a proximal end and a distal end
incorporating a biocompatible material made from an echogenic material within the elongated sheath, wherein the echogenic material surrounds the central lumen; and
attaching a connection fitting to the proximal end of the elongated sheath.

### BRIEF DESCRIPTION OF THE DRAWINGS

FIG. 1 is a side view of a catheter;

FIG. 2 is a side view of the catheter with an inserted electrostimulation needle;

FIG. 3 is a side view of the catheter with incorporated echogenic particles;

FIG. 4 is a cross-sectional view of the catheter with incorporated echogenic particles;

FIG. 5 is a side view of the catheter with incorporated echogenic strips;

FIG. 6 is a side view of the catheter with an incorporated echogenic coil;

FIG. 7 is a side view of the catheter with an attached thin echogenic layer;

FIG. 8 is a side view of the catheter with attached echogenic printed symbols;

FIG. 9 is a side view of a catheter with a partially inserted electrostimulation needle; and

FIG. 10 is a side view of the catheter with an attached fluid line.

Corresponding reference characters and labels indicate corresponding elements among the view of the drawings. The headings used in the figures should not be interpreted to limit the scope of the claims.

### DETAILED DESCRIPTION

A peripheral nerve block catheter and methods of situating and using the catheter in an anesthetic nerve block procedure are described. The peripheral nerve block catheter described herein overcomes the limitations of previous designs in several significant ways. The catheter includes an elongated sheath having an electrostimulation needle disposed within the elongated sheath. The catheter is constructed of a biocompatible material that further incorporates an echogenic material, thereby rendering the catheter highly visible to ultrasound. In use, the catheter may be situated relative to a nerve to be blocked, using a combination of ultrasound imaging and electrostimulation to elicit motor or sensory responses. Once the catheter is in place, the electrostimulation needle is removed from the sheath, and local anesthetic may be supplied though the catheter with minimal leakage. Because the catheter is situated and inserted in a single step, placement accuracy is enhanced relative to prior catheter designs. In addition, because the material of the catheter incorporates echogenic material, the placement of the catheter may be monitored at any time using ultrasound imaging.

The biocompatible material and echogenic material included in the catheter are further selected and sized to provide added stiffness relative to previous catheter designs, resulting in a catheter that is resistant to kinking during use, including extended use in applications such as the treatment of chronic pain conditions. Because the catheter includes echogenic materials, the condition and/or removal of the catheter may be monitored at any time using ultrasound imaging.

Further, the peripheral nerve block catheter may be used safely and effectively by relatively an inexperienced practitioner with minimal training. The technique of situating the peripheral nerve block catheter is familiar to most medical practitioners due to the similarity of this technique to widely practiced procedures such as intravenous catheter placement. Further, the use of both electrostimulation and ultrasound imaging to guide the placement of the peripheral nerve block catheter provides ample feedback to ensure accurate and effective catheter placement.

Aspects of the peripheral nerve block catheter and methods of using the peripheral nerve block catheter are described in detail below.

### I. Peripheral Nerve Block Catheter

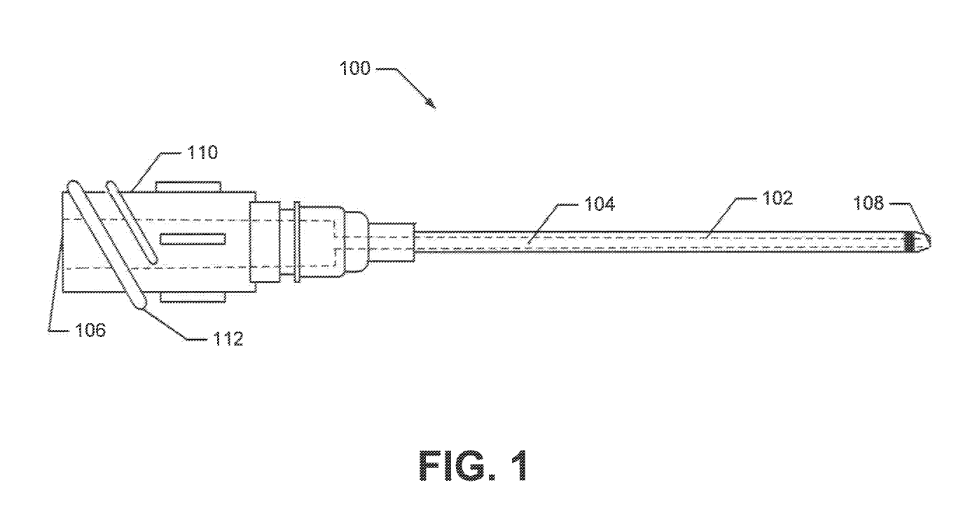

FIG. 1 shows the various structural aspects of the nerve block catheter 100. The catheter 100 comprises an elongate sheath 102 comprising a biocompatible material, which forms a thin wall surrounding a central lumen 104 (shown in phantom line), which is defined along the entire length of the catheter 100. The central lumen 104 communicates with a proximal end 106 and the distal end 108 of catheter 100, thereby forming a continuous conduit for the transport of fluids such as blood, saline, or an anesthetic composition during a regional anesthetic nerve block procedure. The catheter 100 further includes a connection fitting 110 attached at the proximal end 106. The connection fitting 110 may include a raised thread 112 designed to mesh with corresponding grooves of a connector such as the fitting on a fluid line. The distal end 108 may optionally incorporate a tip marker 114 made of an echogenic material to provide the capability to locate the distal end 108 of the catheter 100 using ultrasound imaging techniques during the placement and/or removal of the catheter 100 during a nerve block procedure.

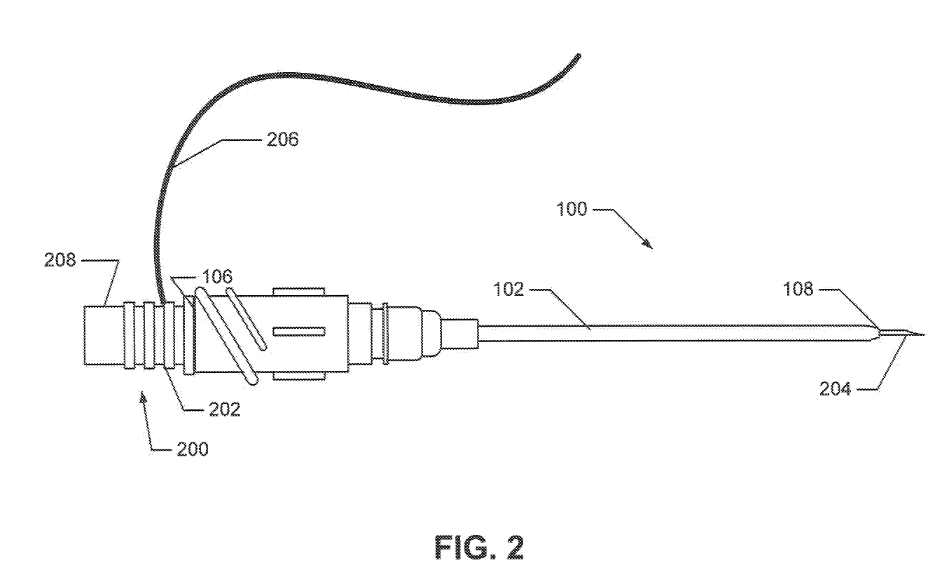

Referring to FIG. 2, the catheter 100 may further include an electrostimulation needle 200 comprising a beveled end 204 and a conductive wire 206 electrically attached at an opposite end 208 of needle 200, which may also include an attachment fitting 202 designed to mechanically lock the needle 200 into the distal end 106 of the catheter 100 using known methods, such as a friction fit or matched threaded fittings. During placement of the catheter 100, the electrostimulation needle may be situated within the cylindrical lumen 104 (FIG. 1) such that the beveled end 204 protrudes from the distal end 108 of the sheath 102.

In an embodiment, the sheath 102 of the catheter 100 may range in length from about 2 cm to about 16 cm. In other aspects, the sheath 102 may range in length from about 2 cm to about 4 cm, from about 3 cm to about 5 cm, from about 4 cm to about 6 cm, from about 5 cm to about 7 cm, from about 6 cm to about 8 cm, from about 7 cm to about 9 cm, from about 8 cm to about 10 cm, from about 9 cm to about 11 cm, from about 10 cm to about 12 cm, from about 11 cm to about 13 cm, from about 12 cm to about 14 cm, from about 13 cm to about 15 cm, and from about 14 cm to about 16 cm. The length of the sheath 102 may be selected based on the particular nerve block procedure and/or type of patient to be anesthetized using a nerve block. For example, shorter sheaths may be used in nerve block procedures involving catheter placement through the neck or shoulder girdle region, and longer sheaths may be used in nerve block procedures involving catheter placement through the belly or leg. In addition, a longer sheath may be indicated for use in obese patients, and a shorter sheath may be used to perform a nerve block procedure in a non-obese patient.

The catheter sheath 102, connection fitting 110, electrostimulation needle 202, and methods of using the catheter 100 to perform a nerve block procedure are described in detail below.

a. Sheath

The sheath 102 of the nerve block catheter 100 may incorporate biocompatible materials as well as echogenic materials. The materials and design of the sheath 102 result in at least several key functional features that enhance the efficacy of the regional anesthetic nerve block catheter 100. The inclusion of echogenic materials in the sheath 102 result in enhanced contrast between the catheter 100 and surrounding biological tissues in ultrasound images obtained during the insertion, removal, repositioning and monitoring of the catheter 100 during use in a nerve block procedure. Further, both biocompatible and echogenic materials of the sheath 102 may impart structural stiffness to resist bending and compressive stresses, enhancing the resistance of the sheath 102 to kinking during use resulting from disturbance of the catheter due to factors such as patient movements. However, the structural stiffness of the sheath 102 should not be so stiff as to result in tissue damage induced by disturbances of the catheter 100 induced by patient movements.

The resistance to kinking may be achieved by any one or more of at least several approaches including but not limited to: selection of a stiff biocompatible and/or echogenic materials, the use of thickened sheath walls, and any combination thereof.

i. Biocompatible Materials

The biocompatible materials suitable for use in the construction of the sheath 102 may be any biocompatible material known in the art so long as the material is sufficiently flexible to minimize the risk of tissue injury resulting from movements or repositioning of the catheter 100 when inserted in the patient, yet sufficiently stiff to enhance the resistance of the catheter 100 to kinking. Non-limiting examples of suitable biocompatible materials include silicone, polypropylene, polyethylene, polysulfone, polyethersulfone, polyvinylidene difluoride, polycarbonate, nylon, polyamide, PTFE (Teflon), high density polyethylene, urethane and any combination thereof. In addition, the biocompatible materials may further include reinforcing materials such as metal, silk or collagen fibers to enhance the stiffness of the biocompatible materials.

In one aspect, the thickness of the sheath wall surrounding the central lumen 104 may range from about 0.2 mm to about 2 mm. In other aspects, the thickness of the sheath wall surrounding the central lumen 104 may range from about 0.2 mm to about 0.4 mm, from about 0.3 mm to about 0.5 mm, from about 0.4 mm to about 0.6 mm, from about 0.5 mm to about 0.7 mm, from about 0.6 mm to about 0.8 mm, from about 0.7 mm to about 0.9 mm, from about 0.8 mm to about 1.0 mm, from about 0.9 mm to about 1.1 mm, from about 1.0 mm to about 1.2 mm, from about 1.1 mm to about 1.3 mm, from about 1.2 mm to about 1.4 mm, from about 1.3 mm to about 1.5 mm, from about 1.4 mm to about 1.6 mm, from about 1.5 mm to about 1.7 mm, from about 1.6 mm to about 1.8 mm, from about 1.7 mm to about 1.9 mm, and from about 1.8 mm to about 2.0 mm. The thickness of the sheath walls may vary depending on the strength of the biocompatible materials included in the sheath 102, the length of the catheter 100, the diameter of the lumen 104 within the sheath 102, the desired stiffness of the sheath 102, and the inclusion of echogenic materials, which may also serve as reinforcing materials, in the walls of the sheath 102.

ii. Echogenic Materials

The catheter 100 may include echogenic materials incorporated into the sheath 102 in order to enhance the contrast of the sheath 102 relative to surrounding biological tissues in an ultrasound image of the catheter inserted in the vicinity of a nerve in a nerve block procedure. In addition, the echogenic materials may also enhance the structural stiffness of the catheter 100, thereby imparting increased resistance to pinching or kinking.

Any echogenic material known in the art may be included in the sheath 102 of the catheter 100. Non-limiting examples of echogenic materials include metals and metal alloys such as stainless steel, nickel-titanium alloy, copper alloy, platinum, iron, and any combination thereof. The echogenic material may be incorporated in any one of at least several ways illustrated below.

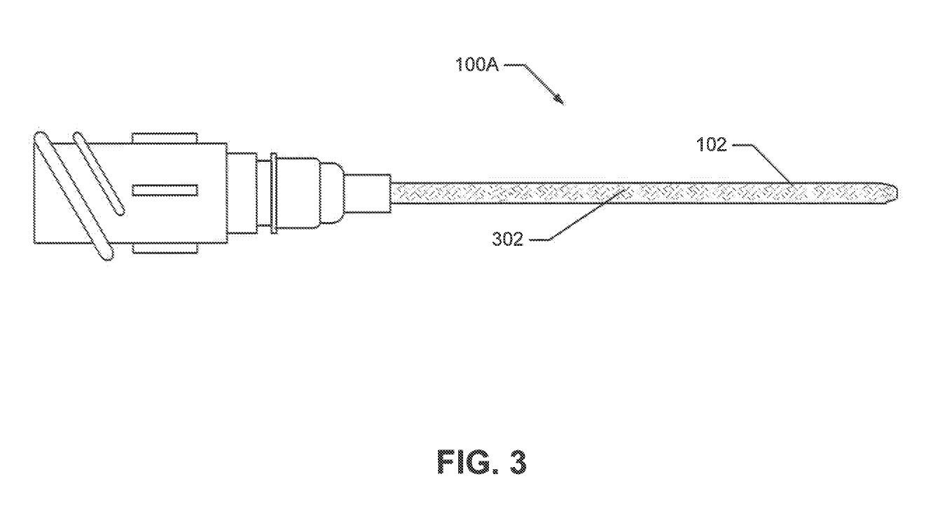

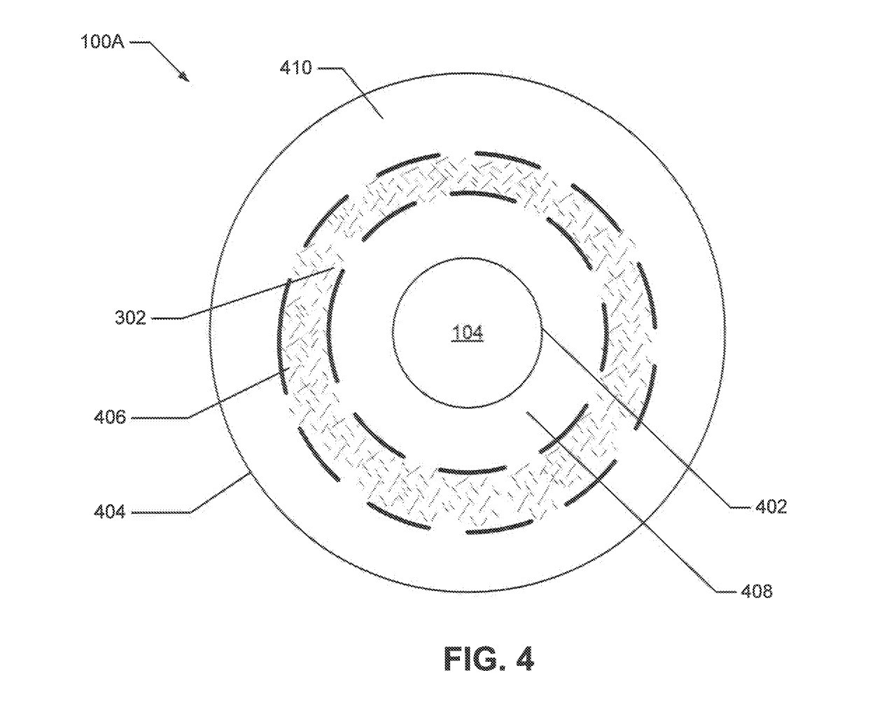

Referring to FIG. 3, an embodiment of the catheter, designated 100A, may incorporate the echogenic material into the sheath 102 in the form of echogenic particles 302 situated within the wall of the sheath 102. The echogenic particles 302 may be situated such that the particles 302 are not directly exposed to either the inner lumen surface 402 or the outer sheath surface 404, as shown in FIG. 4. In this aspect, the echogenic particles 302 may be interspersed within a central sheath layer 406, which is sandwiched between an inner layer 408 and an outer layer 410, both of which may be composed essentially of biocompatible materials.

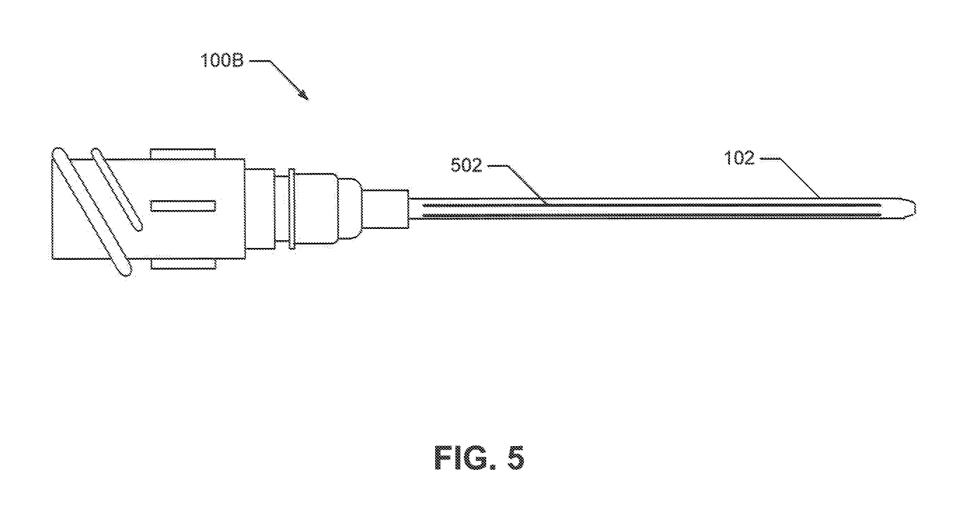

Alternatively, as shown in FIG. 5 the echogenic material may be incorporated in the form of discrete strips, wires, or bars, as shown in the catheter, designated 1008. In this aspect, the echogenic material is provided in the form of one or more strips 502 imbedded in the biogenic material of the sheath 102. The one or more strips 502 may be aligned along the longitudinal axis of the catheter 102 and may be defined along the entire length of the sheath 102, as illustrated in FIG. 5. Alternatively, the strips 502 may have a length that is shorter than the length of the sheath 102 such that more than one strip 502 may be aligned end-to-end to extend the length of the sheath 102, or the shorter strips 502 may be staggered circumferentially around outside of the sheath 102 as well as longitudinally along the length of the sheath 102. Other distribution patterns and strip lengths are also contemplated.

The one or more strips 502 may have any known symmetrical or non-symmetrical cross-sectional shape including but not limited to circular, elliptical, tear-drop, I-beam, tubular, square, rectangular, and polygonal. The cross-sectional dimensions are selected to result in a strip 502 that may be imbedded within the biocompatible material of the sheath 102, in a manner similar to that shown in FIG. 4. The one or more strips 502 may be straight along the length of each strip, as shown in FIG. 5, or the one or more strips 502 may have a curved or bent shape, including by not limited to an arc, a sinusoidal curved shape, one or more acute angle bends along the length including a zigzag shape, and an combination thereof. For example, the one or more strips 502 may have a bent shape to provide enhanced resistance to bending and kinking in one direction, but more flexibility in bending in another direction, if so desired.

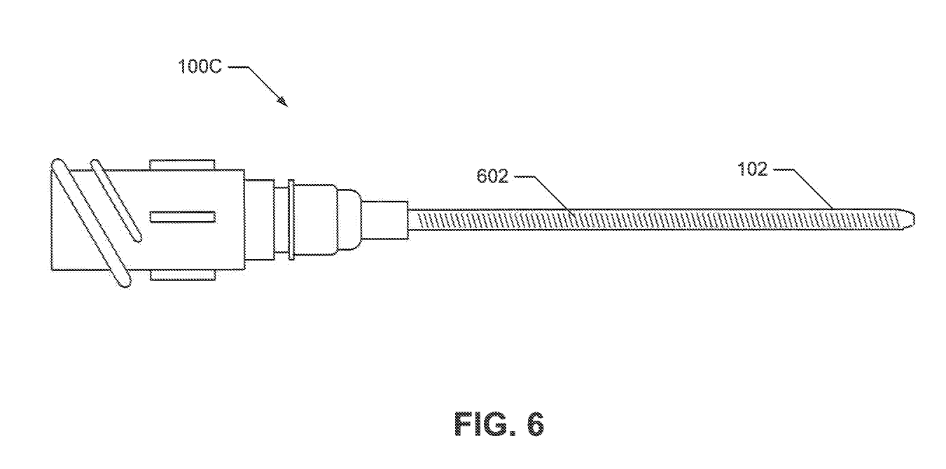

In one aspect as shown in FIG. 6, the catheter, designated 100C, may incorporate echogenic material in the form of a coil 602. The coil 602 may be a continuous strip or wire in a spring shape extending along the length of the sheath 102 in a helical pattern. The helical pattern may include any number of turns, from about a quarter turn along the length of the sheath 102 to a plurality of turns along the length of the sheath 102, as illustrated in FIG. 6. Alternatively, the coil 602 may include two or more continuous strips in which each strip forms a helical pattern running up to the entire length of the sheath 102. In addition, the two or more continuous strips may be formed into helical patterns having different coil angles, defined herein as the angle between the coil and the longitudinal axis of the sheath 102. Combinations of different coil angles may result in a multiple helix pattern or a helical mesh-like pattern. The coil angle may be uniform along the length of the sheath 102, or the coil angle may become steeper or flatter along the length of the sheath 102. For example, the coil angle may be very steep near the ends of the catheter 100 and flatten near the middle of the catheter 100, resulting in a catheter 100 with stiff ends and a flexible middle section, if so desired.

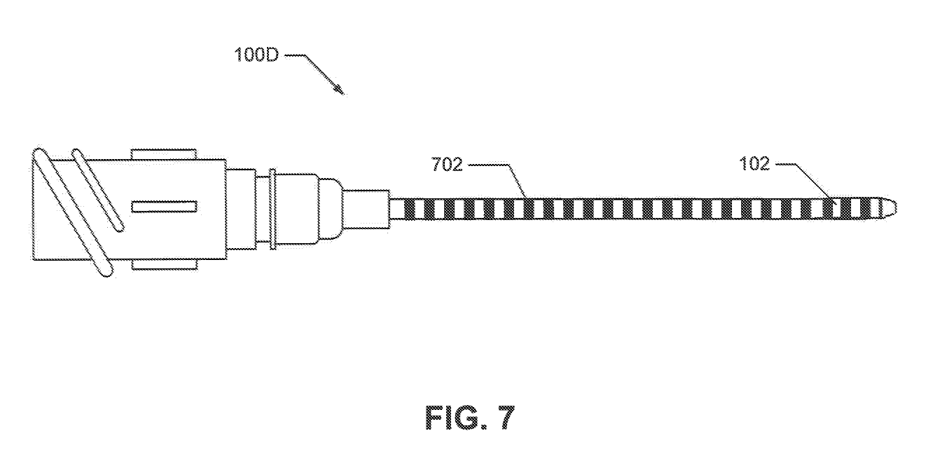

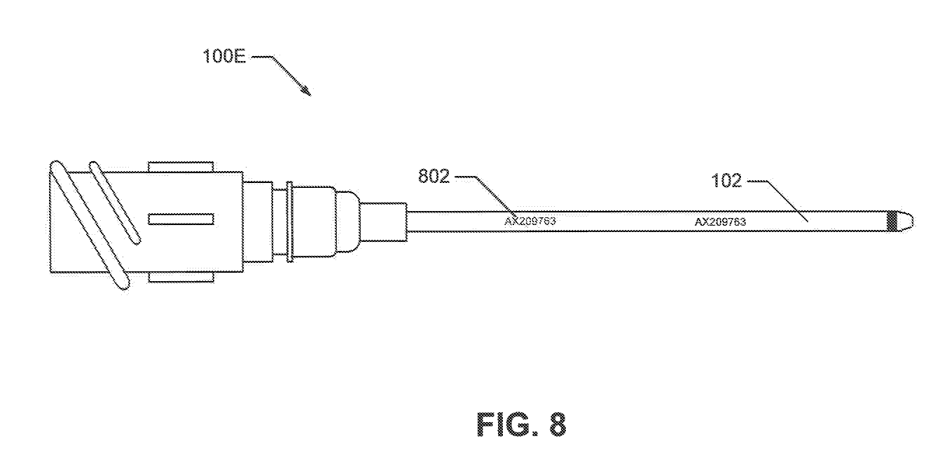

In yet another aspect, a catheter, designated 100D, may incorporate an echogenic material in the form of a relatively thin echogenic layer 702 attached to the outer surface 404 of the sheath 102, as shown in FIG. 7. In this aspect, the echogenic material may be attached to the outer surface using any known method, including but not limited to sputter deposition, ion plating, sol-gel techniques, printing or stamping using metallic paints or dyes, and any other technique known in the art. The pattern of the echogenic layer 702 may be any arbitrary pattern, and may occupy up to the entire outer surface area of the sheath 102. For example, the echogenic layer 702 may be provided in the form of a series of rings. In another example, shown in FIG. 8, a catheter, designated 100E, may include echogenic printed symbols 802 attached to the sheath 102 to indicate information. Non-limiting examples of information that may be indicated by the echogenic printed symbols includes: the model number of the catheter 100, the date of the nerve block procedure, a patient identification number, the depth of insertion of the catheter 100, and the orientation of the catheter 100.

b. Electrostimulation Needles

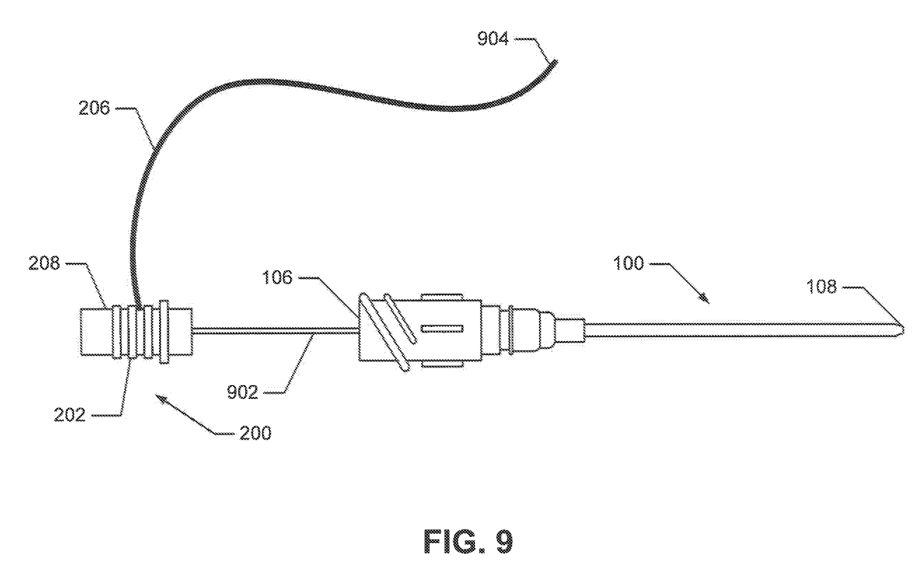

Referring to FIG. 9, an electrostimulation needle 200 may be inserted into the catheter 100 by slipping the needle shaft 902 of the needle 200 into the lumen opening (not shown) at the proximal end 106 of the catheter 100 until the beveled end 204 (FIG. 2) of the needle 200 emerges from the distal end 108 of the catheter 100. FIG. 2, discussed previously, illustrates the needle 200 inserted fully into the catheter 100. The needle 200 provides additional structural stiffness and a sharpened beveled point in order to facilitate the insertion and placement of the catheter 100 in the proximity of a nerve in a nerve blocking procedure.

The electrostimulation needle 200 may be any electrostimulation needle known in the art, including but not limited to injection needles that may incorporate an internal fluid channel for the injection of liquid substances, as well as solid cross-section needles. The needles may be of any design including uninsulated needles as well as coaxial needles incorporating an insulative coating along the shaft of the needle except for an exposed metal tip. In addition, the electro-stimulation needle 200 may be constructed of any biocompatible and electrically conductive material known in the art. Non-limiting examples of suitable materials for the construction of the electrostimulation needle 200 include stainless steel, titanium, nickel, and any combination thereof.

The electrostimulation needle 200 may further include a conductive wire 206 that is electrically connected to the needle shaft 902 such that electrical current may be delivered to the tissues of the patient through the beveled end 204 (FIG. 2). The electrical current may be supplied by any known electrostimulator known in the art, which may be electrically attached to the conductive wire 206 at its free end 904. The conductive wire 206 may be electrically connected to the needle shaft 902 using any known method known in the art including but not limited to welding, soldering, crimping, clamping, adhesion with conductive glue, or any other method known in the art. The conductive wire 206 may be electrically connected at any location along the shaft 902. In a preferred aspect, the conductive wire 206 may be electrically connected to the shaft 902 through a passage in the attachment fitting 202, as shown in FIG. 9.

The needle 102 of the catheter 100 may range in length from about 2 cm to about 20 cm. In other aspects, the needle 102 may range in length from about 2 cm to about 4 cm, from about 3 cm to about 5 cm, from about 4 cm to about 6 cm, from about 5 cm to about 7 cm, from about 6 cm to about 8 cm, from about 7 cm to about 9 cm, from about 8 cm to about 10 cm, from about 9 cm to about 11 cm, from about 10 cm to about 12 cm, from about 11 cm to about 13 cm, from about 12 cm to about 14 cm, from about 13 cm to about 15 cm, and from about 14 cm to about 16 cm. The length of the needle 102 may be selected based on the particular nerve block procedure and/or type of patient to be anesthetized using a nerve block, as discussed above.

The diameter of the electrostimulation needle 200 may range between about 24 gage to about 16 gage. In other aspects, the diameter of the electrostimulation needle 200 may be 20 gage, 19 gage, 18 gage, 17 gage, or 16 gage. Referring back to FIG. 2, the beveled end 204 of the needle 200 may have any known bevel angle, and typically this bevel angle may range between about 15° and about 60°. In other aspects, the bevel angle of the electrostimulation needle 200 may range between about 15° and about 25°, between about 20° and about 30°, between about 25° and about 35°, between about 30° and about 40°, between about 35° and about 45°, between about 40° and about 50°, between about 45° and about 55°, and between about 50° and about 60°.

The diameter and bevel angle of the needle 200 may be selected based one or more of at least several factors including the minimization of the size of the entry wound, the minimization of tissue and or nerve damage during catheter placement, the tactile feedback during the insertion of the catheter 100, and any combination thereof. For example, the use of a thinner, sharper needle may minimize the size of the entry wound, but may also increase the chances of tissue and/or nerve injury during the insertion of the catheter 100. Conversely, the use of a thicker, blunter needle may enhance the tactile feedback to the practitioner and decreases the chance of injury to the nerve and/or intervening tissues during the insertion of the catheter 100, but may also result in a larger entry wound. In one aspect, more than one needle may be used during the insertion of the catheter 100 during a nerve block procedure, as discussed in detail below.

### II. Methods of Using Peripheral Nerve Block Catheter

The catheter 100 may be used to perform a novel regional anesthetic nerve block procedure in which both ultrasound and electrostimulation are used to accurately locate the catheter in close proximity to the nerve to be blocked, due the inclusion of both an electrostimulation needle 200 as well as echogenic materials in the design and construction of the catheter 100. A detailed description of various aspects of a method of performing an anesthetic nerve block using the catheter 100 is provided below.

The catheter 100 may be used to perform any type of anesthetic nerve block performed by existing nerve block catheters. For example, the type of nerve block that may be performed using the catheter 100 may include an upper extremity block, a truncal block, and a lower extremity block.

Non-limiting examples of upper extremity blocks include nerve blocks in the area of the brachial plexus including interscalene blocks, supraclavicular blocks and axillary blocks. Non-limiting examples of truncal blocks include nerve blocks in the area of the thoracic and lumbar nerves including the paravertebral block, intercostal block, transversus abdominis plane block and rectus sheath block. Non-limiting examples of lower extremity blocks include nerve blocks in the area of the lumbar plexus including sciatic blocks, femoral blocks, lateral femoral blocks, obturator blocks, popliteal blocks, ankle blocks, and lumbar sympathetic block; and nerve blocks in the area of the celiac plexus including celiac plexus blocks, which block the splanchnic nerve bundle. Another non-limiting example of a nerve block, which may be performed using the catheter 100, is a continuous epidural anesthesia procedure in which the catheter may be inserted into a patient's epidural space between T1 (thoracic vertebra number 1) through S5 (sacral vertebra number 5) to block impulse from the spinal nerves in the T10 through S5 region.

The method of using the catheter 100 to perform a nerve block procedure shares many similarities to existing methods during preparation of the patient with respect to location and marking of the catheter insertion point and sterilization of the patient in the vicinity of the entry point. However, because the catheter insertion process using the catheter 100 as described above is a one-step process with no separate catheter insertion step, only a small area of about 5-10 cm² around the catheter insertion point need be sterilized, compared to significantly larger area required by existing nerve block procedures. In addition, no sterilization drape is necessary nor is a sterile ultrasound probe cover required.

Prior to catheter insertion, an ultrasound imaging device may be used to locate the nerve to be blocked and left in place to visualize the position and movements of the catheter during the subsequent insertion of the catheter. To prepare the catheter for insertion, an electrostimulation needle 200, with a conductive wire 206 connected to an electrostimulator, is inserted into the lumen 104 of the catheter 100, resulting in the arrangement shown in FIG. 2.

Once the catheter 100 and electrostimulation needle 200 have been assembled, the beveled end 204 may be situated on the marked entry site and used to puncture through the epidermis to initiate the placement of the catheter 100. Ultrasound imaging may be used to monitor the movements of the catheter to the nerve to be blocked through the intervening tissues, using the ultrasound image to avoid the injury of any vulnerable tissues such as blood vessels. Once the catheter 100 is thought to be sufficiently near the nerve to be blocked, the electrostimulator may be used to introduce stimulating electrical currents to the tissues through the beveled tip 204 until a sensory or motor response characteristic of the nerve to be blocked is observed. For example, the twitching of a patient's fingers or arm may be observed in the case of a block in the vicinity of the brachial plexus.

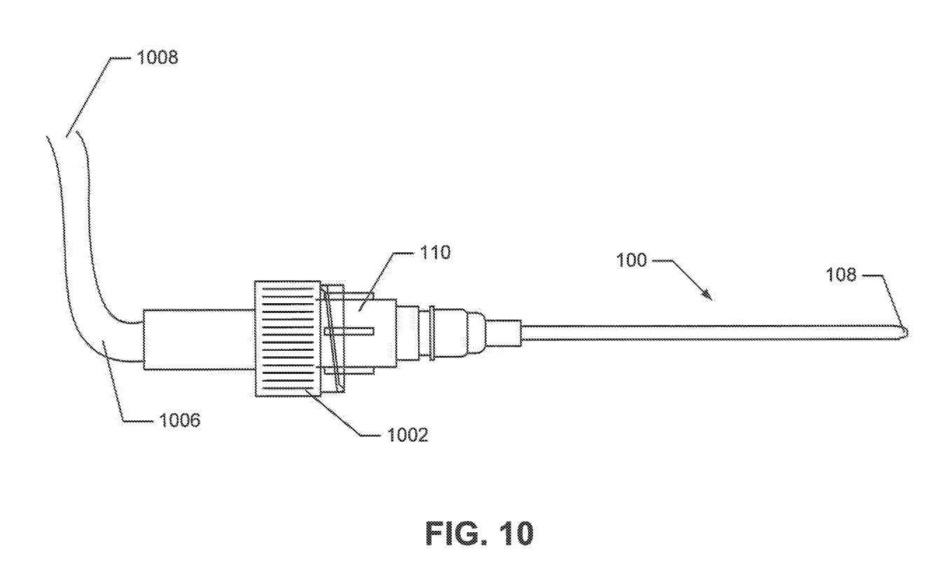

Once a response to electrostimulation is observed indicating appropriate placement of the catheter 100, an injection of a small amount of an appropriate anesthetic compound through the electrostimulation needle 200 may be administered such that the spread of the anesthetic compound is observed under ultrasound and a diminished response to stimulation may also be observed in order to verify proper placement of the catheter 100, assuming a hollow injection-type electrostimulation needle 200 was used. Once the proper placement of the catheter 100 has been verified, the electrostimulation needle 200 may be removed from the catheter 100, and a fluid line 1006 may be attached to the connection fitting 110 of the catheter 100, as shown in FIG. 10. The fluid line 1006 may be attached to the catheter 100 by any known method, including a threaded receptacle 1002 that fits over the connection fitting 110, as shown in FIG. 10. Additional anesthetic compound may be introduced into catheter 100 through the fluid line 1006 as needed to deliver the anesthetic compound from the distal end 108 into the region adjacent to the nerve to be blocked. If a solid electrostimulation needle 200 was used in this method, the initial injection of the anesthetic compound through the needle 200 may be skipped, and instead the anesthetic compound may be introduced through the catheter 100 after removing the electrostimulation needle 200 by way of a connected fluid line 1006 as described previously.

The free end 1008 of the fluid line 1006 may be attached to any known means of delivering liquid anesthetic formulations including but not limited to fluid infusion pump, patient controlled anesthetics delivery device, hypodermic syringes, pressurized fluid containers, and drip bags. If the catheter 100 is to be retained by the patient for the long-term relief of a chronic pain condition, the free end 1008 of the fluid line 1006 may be connected to an existing device that may introduce an amount of an anesthetic compound according to a predetermined schedule, which may be modified by patient inputs.

In another aspect, more than one needle may be used in a method of performing a nerve block using the catheter 100. In this aspect, a thinner, sharper needle 200A may be used to perform the initial insertion of the catheter 100 into the patient. Once the catheter 100 has been advanced a short distance, the thinner, sharper needle 200A may be removed from the catheter 100 and replaced by a thicker, sharper needle 200B. In this aspect, the initial use of the thin, sharp needle 200 results in a smaller entry wound, while the subsequent use of the thicker, blunter needle 200 provides enhanced tactile feedback and minimizes damage to the tissue, nerves and blood vessels, while advancing the catheter 100 through the intervening tissues to the vicinity of the nerve to be blocked.

As discussed above, ultrasound imaging may be used to monitor the condition of the catheter 100 during the initial nerve block procedure. In addition, if the catheter 100 is to be removed at the completion of an associated surgical procedure, ultrasound imaging may be used to monitor the condition of the catheter during the course of the surgical procedure, as well as to ensure that the entire catheter was removed intact at the completion of the surgical procedure. In addition, if the catheter 100 is to be retained by the patient for an extended period, ultrasound imaging may be used to periodically monitor the condition of the catheter 100 and repositioning the catheter 100 may be accomplished by inserting the thicker, blunter needle 200 into the catheter 100 to reposition the catheter 100, as well as to ensure that the catheter 100 is removed intact upon removal.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A catheter comprising:
an elongated sheath defining a central lumen in communication with a proximal end and a distal end,
an echogenic material made from a biocompatible material surrounding the central lumen, and
a connection fitting attached to the proximal end of the elongated sheath.

2. The catheter of claim 1, wherein the central lumen has an internal diameter sized to accommodate a needle diameter ranging from about 24 gage to about 16 gage.

3. The catheter of claim 1, wherein the catheter has a length ranging from about 2 cm to about 20 cm.

4. The catheter of claim 1, wherein the echogenic material comprises a metal or metal alloy comprising at least one of a stainless steel material, a nickel-titanium alloy material, a copper alloy material, a platinum material, and an iron material.

5. The catheter of claim 1, wherein the echogenic material is embedded within the biocompatible material such that the echogenic material is not exposed to the central lumen or to an exterior surface of the elongated sheath.

6. The catheter of claim 1, wherein the echogenic material is attached to an exterior surface of the elongated sheath.

7. The catheter of claim 1, wherein the biocompatible material comprises at least one of a silicone material, a polypropylene material, a polyethylene material, a polysulfone material, a polyethersulfone material, a polyvinylidene difluoride material, a polycarbonate material, a nylon material, a polyamide material, a PTFE material, a high density polyethylene material, and a urethane material.

8. The catheter of claim 1, further comprising:
an electro-stimulation needle comprising a beveled end and a conductive wire electrically connected at an opposite end thereof, wherein the electrostimulation needle is disposed within the central lumen such that the beveled end protrudes out of the distal end of the central lumen.

10. The catheter of claim 8, wherein the electrostimulation needle is constructed of an electrically conductive and biocompatible material chosen comprising at least one of a stainless steel material, a titanium material, and a nickel material.

11. The catheter of claim 8, wherein the electrostimulation needle comprises at least one of an injection needle and a solid cross section needle.

12. A method for manufacturing a catheter comprising:
forming an elongated sheath defining a central lumen with a proximal end and a distal end
incorporating a biocompatible material made from an echogenic material within the elongated sheath, wherein the echogenic material surrounds the central lumen; and
attaching a connection fitting to the proximal end of the elongated sheath.

13. The method of claim 12, further comprising disposing an electro-stimulation needle within the central lumen.

14. The method of claim 13, wherein the electro-stimulation needle includes a beveled end and a conductive wire electrically connected to a portion of the electro-stimulation needle, and wherein inserting the electro-stimulation needle further comprises disposing the electro-stimulation needle within the central lumen such that the beveled end protrudes outwardly from the distal end of the central lumen.

15. The method of claim 12, wherein the echogenic material is in the form of at least one or more coils, one or more of continuous strips, one or more layers, one or more strips, one or more bars, and combinations thereof.

16. The method of claim 12, wherein the echogenic material comprises at least a metal or metal alloy comprising at least one of a stainless steel material, a nickel-titanium alloy material, a copper alloy material, a platinum material, and an iron material.

17. A method of using a catheter comprising:
providing a catheter comprising:
an elongated sheath defining a central lumen in communication with a proximal end and a distal end,
an echogenic material made from a biocompatible material surrounding the central lumen, and
a connection fitting attached to the proximal end of the elongated sheath;
inserting an electro-stimulation needle comprising a beveled end and a conductive wire electrically connected at an opposite end thereof through the central lumen such that the beveled end protrudes outwardly from the distal end of the central lumen; and
puncturing a body wall with the beveled end of the electro-stimulation needle;
positioning the catheter such that the beveled end is adjacent or proximate a nerve; and
introduce stimulating electrical currents through the electro-stimulation needle.

18. The method of claim 17, further comprising injecting an anesthetic compound through the electro-stimulation needle.

19. The method of claim 17, further comprising removing the electro-stimulation needle from the central lumen after introduction of the stimulating electrical currents.

20. The method of claim 19, further comprising injecting an anesthetic compound through the central lumen of the elongated sheath after removal of the electro-stimulation needle.
